(19) United States
(12) Patent Application Publication
(10) Pub. No.: US 2006/0204560 A1
(43) Pub. Date: Sep. 14, 2006

(54) Wound dressing consisting of a biodegradable biopolymer

(76) Inventors: Alexander John Walacavage, Lansing, MI (US); John Francis Holland, Lansing, MI (US)

Correspondence Address: JOHN F. HOLLAND, 2411 LYMAN DRIVE, LANSING, MI, 48912, US

(21) Appl. No.: 11/433,607

(22) Filed: May 15, 2006

Related U.S. Application Data
(63) Continuation-in-part of application No. 10/675,722, filed on Sep. 30, 2003.
(60) Provisional application No. 60/414,255, filed on Sep. 30, 2002.

(51) Int. Cl. A61K 31/718; A61L 15/00

(52) U.S. Cl. 424/445; 514/60

(57) ABSTRACT

A hydrophilic pliable wound dressing consisting of biopolymers creates a new approach to wound dressing by providing a self-assembling hydrophilic gel type moisture permeable barrier applicable to a wide range of wound sizes and depths. Biodegradability and water solubility enable rapid and safe disposal, obviating a significant problem that occurs with conventional type dressings. The initial sheet form of the dressing provides an ideal carrier for delivery of nutrients, debriders, antibiotics, analgesics and physiological regulators.

This application is a continuation-in-part of application # 10/675,722 filed on Sep. 30, 2003 which has a priority to Provisional Application # 60/414,255 filed on Sep 30, 2002.

There is no Federal Funding associated with any aspect of this invention.

### SPECIFICATION

### Background of the Invention

Wound healing presents a challenge to medical and surgical personnel. This is particularly true with large wounds having poorly defined edges; for example, burns, decubitus ulcers, venous stasis ulcers, arterial ulcers and serious abrasions. Wounds are often treated by covering them with products such as alginates, composites, contract layers, foams, hydrocolloids, hydrogels, impregnated gauzes, specialty absorptive, and transparent films. The theory behind the use of these products is that covering the wound decreases the risk of infection, keeps the wound from drying out, and decreases scarring.

Numerous products have been introduced which theoretically increase the rate of wound healing. Examples include topical cleansers, sealants, protectants, moisturizers, and enzymatic debriding agents. More recently, biological and biosynthetic dressings impregnated with collagen or related hydrolysates have been introduced. The theory behind these agents is that the collagen contained within the dressings mimics the natural collagen produced by the body and, thereby, promotes healing. Without treatment secreting skin wounds may lead to anemia, infections, shock and even death through the loss of body proteins, electrolytes, fluids and heat. However, all of these approaches have shortcomings related to absorption characteristics and capacities, wound cleaning efficiency, discomfort in application and removal, bacterial susceptibility, expense and, in many cases, disposal problems. The unique proposed dressing consisting of a homogenous biodegradable polymer, such as starch foam, addresses and eliminates many of these shortcomings providing a self assembling gel barrier, which absorbs large quantities of exudates, maintains appropriate levels of hydration and allows body movement without restraint. The homogenous nature of the starch foam has a double meaning. Firstly, the extruded foam is of one type and consistency and the second meaning comes from the fact that the foam is used by itself with no other adducts that are typically added to provide structural lattice, isolation, conductivity, protection or multi-functionality. The homogeneity and biodegradability give this dressing a uniqueness in the field.

There are two types of degradability, environmental and biological. Environmental includes any chemicals or forces acting on the material and dissolving or eroding its structure, which usually takes a very long time. Biological or biodegradation occurs as the result of living organisms acting on the material. Degradation here takes a much shorter period of time and often can result in usable by products. Recently, the term biodegradable is receiving much attention and usage, most of which is inappropriate and confusing and does little to define the real nature of a material. The American Standards for Testing and Measurement (ASTM) is in the process of providing definitions for biodegradability based on degradation efficiency and time. For foam such as the product of this application it should be 60-80 or more percent degraded over a 6-month period. Due to the relative solubility of our foam, dispersing in water greatly shortens this time and removes the material from sites where it could aggregate. Indeed, this dressing is nearly 100% degradable and with water rinsing, immediately disposable, obviating a real problem that occurs in most medical facilities or situations. We can honestly use the term, biodegradable.

### SUMMARY OF THE INVENTION

A hydrophilic pliable wound dressing consisting of a homogenous biopolymer provides a self-assembling hydrophilic gel serving as a moisture permeable barrier applicable to a wide range of wound sizes and depths. Biodegradability and water solubility enable rapid and safe disposal, obviating a significant problem that occurs with conventional type dressings. The initial sheet form of the dressing provides an ideal carrier for delivery of nutrients, enzymatic debriders antibiotics, analgesics and physiological regulators.

### BRIEF DESCRIPTION OF THE DRAWINGS

The drawings are simple and self-explanatory.

FIG. 1 shows the nature of the rolls obtained commercially and the sheets later cut from a roll which can be further reduced to approximate wound sizes.

FIG. 2 illustrates a band-aid type embodiment of the dressing.

FIG. 3 illustrates a tubular dispenser that can be used to apply a slightly hydrated form of the biopolymer to open wounds.

### DESCRIPTION OF THE INVENTION

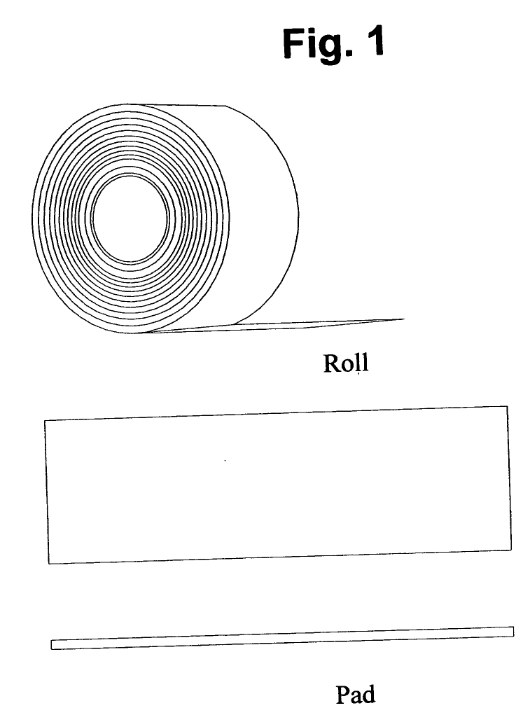

Natural carbohydrate polymers can be extruded in a foam form in the shape of thin sheets and are commercially available. As shown in FIG. 1, these sheets may range in thickness from 1/32 inch to over an inch and in width ½ inch to 32 inches. As shown in this Figure, these sheets may be cut into various sizes and shapes to provide a wide range of dressings for a variety of wound sizes. These sizes can vary from one to a few inches on the sides of square or rectangular shapes to full body or appendage coverings of 1 to a few feet on each side.

For this application the extruded foam sheets of specified formulations appropriate to our needs were obtained commercially. The sheets were then cut to various sizes to accommodate anticipated wound sizes. Subsequent to cutting and sizing, the dressings are packaged antiseptically and made sterile.

In application, the wound to be treated is thoroughly cleansed with sterile water or saline, or a preferred topical agent, and any excess exudates removed and the wound debrided, if necessary. An appropriate sized dressing is removed from its sterile package and placed over the wound. Larger sized dressings can be reduced in size to effectively match the area of the wound exposure. Upon contact with the moisture on the wound tissue, the foam absorbs fluid from the wound turning into a gel-like form creating a protective barrier that provides a moist environment for healing, absorbs drainage, exhibits chemo tactic action and provides topical nutrients. Upon removal, the dressing can be easily and rapidly dissolved by flushing with water and disposed of in a conventional drain.

In one embodiment of the invention, the biopolymer is composed of cornstarch polymerized with the assistance of an agent, such a poly vinyl alcohol (PVA). Other types of starch, such as potato, wheat, rice, etc. may be used as well as other polymerizing agents, such as poly ethylene glycol (PEG), poly propylene glycol (PPG), or one of several natural gums. In a preferred embodiment, the starch used is high amylose cornstarch (National Starch) extruded in flat sheet forms in a process using PVA as the polymerizing agent in a formulation appropriate to our needs; 3% water/90% starch/7% PVA. Where desired, the hydrophilic nature can be controlled by addition of biodegradable polyester (BASF). In the preferred embodiment, packaging is accomplished with commercial packaging wrappers and sterilization of the packaged dressing is accomplished by gamma radiation at levels sufficient for effective sterilization.

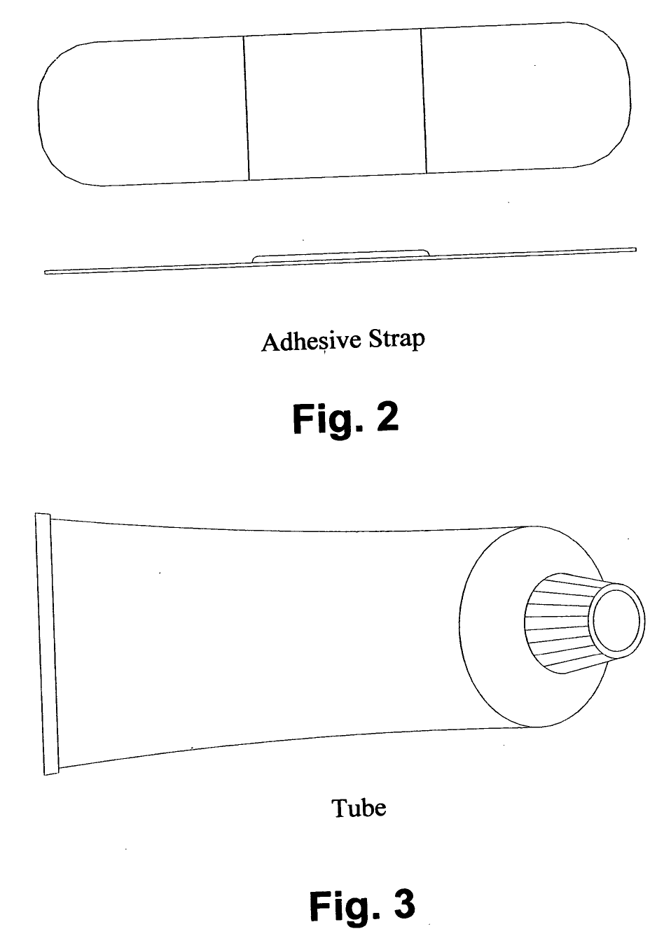

In another embodiment as shown in FIG. 2, the dressings are small and backed by an adhesive tape creating a novel band-aid type bandage that will provide the advantages of the biopolymer dressing to these applications.

In normal situations adequate moisture is present in the wound to convert the polymer foam to the functioning gel. In situations where the natural moisture of a wound is very small, moisturizing the dressing with sterile saline or other agent prior to application may be required for convenience in fitting the dressing onto the wound site. In another embodiment of the invention, the starch foam dressing can be moistened to the point of a viscous fluid and applied to wound sites by dipping from an open container, extrusion from a syringe or a flexible tube, as that shown in FIG. 3. In another embodiment of the invention, the moisturizing agent is a pure water solution of nano-crystalline silver accomplishing both the role of hydrating the resulting gel and acting as a bactericide.

Wounds amenable for treatment with these dressings include acute abrasions, lacerations, burns, stage 2-4 pressure ulcers, diabetic ulcers, venous stasis ulcers, arterial ulcers, donor sites of skin grafts, post surgical incisions, appropriate dental applications and external wounds due to physical trauma.

### EXAMPLE 1

A 54-year-old Hispanic male with type 2 diabetes exhibited a diabetic ulcer stage 2 on the medial aspect of the right lower extremity. Using the conventional wet to dry treatments, the wound was non-healing for 12 months. Application of the starch dressing provided the proper environment promoted the healing, which then occurred in less than 4 weeks. During this time the dressing was changed whenever excessive amounts of excaudate were observed.

### EXAMPLE 2

An 11-year-old male child with lacerations to his left hand and forearm was treated with the starch dressing applied under a clear adhesive cover. The dressing was changed when excessive excaudate was observed and healing occurred within less than 1 week.

### EXAMPLE 3

A 45-year old male was treated for a severe skin tear on this right hand. The wound was cleansed with soap and water and the starch dressing applied and held in place with a Kerlix gauze wrap. Dressing was changed every two days and healing occurred within two weeks. Treatment was discontinued when the skin flap reattached.

### EXAMPLE 4

A 48-year old male received anterior lower leg injuries resulting form a fall. Treatment consisted of cleansing wounds with soap and water, applying starch dressings over open wounds covered with 4×4 gauze and secured with tape. Again healing occurred within 1 week.

In all applications there have never been any signs of infection.

### SUMMARY OF THE ADVANTAGES OF THE INVENTION

The advantageous features of this invention include effective linear wicking and the minimizing of pain and discomfort upon application and removal of the dressing. The gels created are non-toxic locally and systemically. The gel controls odors, decreases purulent exudates and are not. absorbed systematically. The starch foams used are amenable to serve as vehicle for transport and delivery of amino acids, ascorbic acid and other nutrients; enzymatic debriders such as bromolin, and pupain; buffering compounds for pH regulation; and antibiotics, analgesics, bactericides and other compounds for treatment. Where appropriate, scented aromatics can also be incorporated into the foam. A major advantage lies in the ease of site removal and dressing disposal by simple washing and flushing procedures.

We claim:

1. A process whereby a homogenous extruded starch foam is used as a dressing for wound treatment.

2. A process of claim 1 wherein the starch is cornstarch.

3. A process of claim 1 wherein the extrusion formulation consists of cornstarch, water and a polymerizing agent.

4. A process of claim 3 wherein the cornstarch is of high amylose content.

5. A process of claim 3 wherein the polymerizing agent is poly vinyl alcohol.

6. A process of claim 3 wherein the extrusion formulation is 90% starch, 7% PVA and 3% water.

7. A process of claim 3 wherein the hydrophilic nature of the resulting foam is optimally controlled by the addition of a biodegradable polyesters.

8. A process of claim 1 wherein the resulting foam dressing is 99% biodegradable.

9. A process of claim 1 wherein the resulting foam dressing can serve as a vehicle. for delivery of chemical and/or biochemical therapeutic agents.

10. A process in which the extruded starch foam of claim 1 is hydrated to the point where the viscosity is sufficient to form a gel.

11. A process of claim 11 wherein the gel is appropriate for application to dry wound beds via a tube, syringe or other method of application.

12. A process of claim 1 whereby a homogenous extruded foam of 90% high amylose corn starch, 7% poly vinyl alcohol and 3% water forms a biodegradable wound dressing which is further capable of conveying therapeutic agents to the wound site itself.
